(19) United States
(12) Patent Application Publication
(10) Pub. No.: US 2019/0345442 A1
(43) Pub. Date: Nov. 14, 2019

(54) SERUM-FREE CULTURE MEDIUM FOR LIMBAL STEM CELLS AND CULTURE METHOD THEREOF

(71) Applicant: Zhongshan Ophthalmic Center, Sun Yat-Sen University, Guangzhou (CN)

(72) Inventors: Hong OUYANG, Guangzhou (CN); Jin ZHU, Guangzhou (CN)

(73) Assignee: Zhongshan Ophthalmic Center, Sun Yat-Sen Universit y, Guangzhou (CN)

(21) Appl. No.: 15/996,541

(22) Filed: Jun. 4, 2018

(30) Foreign Application Priority Data
May 11, 2018 (CN) 201810450967.8

(51) Int. Cl. C12N 5/0797

(52) U.S. Cl. CPC C12N 5/0623; C12N 2500/90; C12N 2501/11; C12N 2501/33; C12N 2500/46; C12N 2500/38; C12N 2500/30; C12N 2500/20; C12N 2500/05; C12N 2500/42; C12N 2500/36

(57) ABSTRACT

A serum-free culture medium for limbal stem cells and a culture method thereof, wherein the serum-free culture medium includes a basic medium and supplements; wherein the supplements include: human recombinant EGF, insulin, 3,3′,5-triiodo-L-thyronine, hydrocortisone, forskolin, manganese sulfate monohydrate, sodium selenite, sodium metasilicate, ammonium metavanadate, nickel chloride hexahydrate, stannous chloride dihydrate, ethanolamine, O-phosphorylethanolamine, ammonium molybdate tetrahydrate, 4-(2-hydroxyethyl) piperazine-1-ethanesulfonic acid, vitamin C, bovine serum albumin, lipid concentrate and serum substitute. The serum-free culture medium according to the present invention is free of fetal bovine serum and any animal-derived ingredient, can provide necessary adequate nutrition and good environment for cell growth and proliferation, effectively replaces the role of serum, realizes favorable cell growth, improves the cell purity and stability, provides quick and stable cell sources for researches on the mechanism of limbal stem cell specificity and transplantation therapy, and has broad clinical application prospects.

### CROSS-REFERENCE TO RELATED APPLICATION

This application claims the priority benefit of China application serial no. 201810450967.8, filed on May 11, 2018. The entirety of the above-mentioned patent application is hereby incorporated by reference herein and made a part of this specification.

### BACKGROUND

### Technical Field

The present invention belongs to the field of stem cell culture technology, and more particularly relates to a serum-free culture medium for limbal stem cells and a culture method thereof.

### Description of Related Art

A corneal limbus is a border of a cornea with a conjunctiva and a sclera, and an identification mark of the cornea is a termination of a Bowman's membrane. The identification mark of the conjunctiva excludes cup-like cells, is about 1-2 mm wide, and only has an epithelial layer and a stromal layer here. Its epithelial cell layer contains 10 layers of cells, which are arranged irregularly and small cylinder-shaped with hyperchromatic nuclei. Its deep stromal cells are a layer of small cylinder-shaped or cuboidal cells with ovoid cell nuclei, are parallel to a surface, are formed in a nipple of a basilar part, and form a special “fence”-like epithelial structure, which contains pigments and rich vascular networks, and is closely associated with a basal membrane.

A corneal epithelium is a single layer of non-keratinized cells arranged in order. A regeneration of corneal epithelial cells is derived from limbal stem cells (LSCs), which play an important role in maintaining a corneal transparency and vision. During the regeneration, the limbal stem cells migrate to a corneal center and are differentiated. A complete process takes about 14 days. LSCs deficiency is a worldwide and disastrous ophthalmic problem, and seriously threatens a visual quality of human. At present, effective treatment measures are lacked. Common causes include eye trauma, alkali burn, immune eye disease and the like, and are clinically reflected in corneal opacity and visual losses. An ophthalmologic examination takes corneal conjunctivalization, neovascularization, corneal scarring, and symblepharon as diagnostic criteria.

At present, traditional treatment measures against a limbal stem cell deficiency include amniotic membrane transplantation and LSCs transplantation. However, the amniotic membrane transplantation may result in corneal epithelial phenotype changes, while the traditional LSCs transplantation needs many tissues, and are vulnerable to iatrogenic injuries. Therefore, neither has achieved desired clinical therapeutic effects. Autologous limbal transplantation is not suitable for patients with binocular limbal diseases. There are rejections in allogenic limbal transplantation. Therefore, in recent years, it has become a hot topic to treat severe corneal lesions with transplantation of limbal stem cells cultured in vitro in combination with penetrating keratoplasty. At present, methods for culture of limbal stem cells mainly includes culture of limbal stem cells using a variety of different basal layers, such as rodent-derived NIH 373 trophoblast, human-derived amniotic membrane, fibrin glue, Myogel, plasma polymer coating, and human recombinant collagen substrate. Each of these methods can be used to obtain epithelial grafts and are successfully used as a supplement for limbal stem cell deficiency to treat ocular surface diseases.

At present, a certain concentration of fetal bovine serum (FBS) has been added to most media of in vitro amplification systems of limbal stem cells in laboratory and clinical studies at home and abroad. Growth of animal cells depends on the presence of serum. Most cells cannot proliferate in ordinary culture mediums without addition of serum. Serum mainly plays a role in providing necessary hormones, growth factors, transfer proteins and other nutrients for cell growth and proliferation, keeping good cell growth status, and promoting cell growth, division and proliferation. However, FBS contains heterogeneous proteins, and has risks of carrying bacteria, viruses, protein infectious diseases or prions. Secondly, FBS is not stably derived, has unspecific ingredients, and has growth-inhibiting ingredients, thereby going against separation and purification of target products such as vaccines and monoclonal antibodies. Furthermore, residual FBS in the product tends to cause vaccinees' anaphylaxis to serum, and fails to contribute to animal experiments or clinical trials. Therefore, the disadvantages of FBS in clinical mass culture of stem cells have been gradually revealed. At present, many scholars have studied substitutes of FBS. Serum-free culture medium has the characteristics of specific ingredients. However, the existing serum-free culture medium has less ideal culture effects, less ideal stem cell proliferation rate, less ideal purity and counts of cultured stem cells, etc. Therefore, seeking a serum-free culture system with stable quality and convenient use is of great significance for clinical application of LSCs.

### SUMMARY

The technical problem of the present invention is to overcome the defects and deficiencies of the prior art, and to provide a serum-free culture medium for limbal stem cells.

Another object of the present invention is to provide use of the serum-free culture medium in culturing limbal stem cells.

A further object of the present invention is to provide an isolation and culture method of limbal stem cells.

The objects of the present invention are implemented by the following technical solution:

A serum-free culture medium for limbal stem cells includes a basic medium and supplements; wherein the supplements include following ingredients by concentration: 10-20 ng/mL of a human recombinant EGF, 5-10 μg/mL of insulin, 1×10⁻⁹-5×10⁻⁹ M of 3,3′,5-triiodo-L-thyronine, 0.2-1 μg/mL of hydrocortisone, 0.5×10⁻⁵-2×10⁻⁵ M of a forskolin, 0.5×10⁻⁹-2×10⁻⁹ M of manganese sulfate monohydrate, 5×10⁻⁷-10×10⁻⁷ M of sodium selenite, 0.1×10⁻³-1×10⁻³ M of sodium metasilicate (i.e., Na2SiO3.9H2O), 3×10⁻⁶⁻8×10⁻⁶ M of ammonium metavanadate, 3×10⁻¹⁰-8×10⁻¹⁰ M of nickel chloride hexahydrate, 3×10⁻¹⁰-8×10⁻¹⁰ M of stannous chloride dihydrate, 3×10⁻⁷-8×10⁻⁷ M of ethanolamine, 3×10⁻⁶-8×10⁻⁶ M of O-phosphorylethanolamine, 1×10⁻⁹-6×10⁻⁹ M of ammonium molybdate tetrahydrate, 2-8 g/L of 4-(2-hydroxyethyl) piperazine-1-ethanesulfonic acid, 20-50 μg/mL of vitamin C, 1%-3% of a bovine serum albumin, 0.5%-2% of a lipid concentrate, and 10%-15% of a serum substitute.

The present invention has established a novel serum-fee culture system, which does not contain fetal bovine serum, and can provide necessary adequate nutrition and good environment for limbal stem cells growth and proliferation with factors capable of promoting LSCs proliferation, such as epidermal growth factor (EGF), insulin, 3,3′,5-triiodo-L-thyronine, hydrocortisone and forskolin, as culture medium ingredients. The supplements can effectively replace serum ingredients through various mechanisms in the process of culturing limbal stem cells, realize favorable cell growth, and significantly improve the purity and counts of limbal stem cells.

Preferably, concentrations of the ingredients in the supplements of the serum-free culture medium are as follows: 10 ng/mL of the human recombinant EGF, 5 μg/mL of insulin, 2.5×10⁻⁹ M of 3,3′,5-triiodo-L-thyronine, 0.4 μg/mL of hydrocortisone, 1×10⁻⁵ M of the forskolin, 1×10⁻⁹ M of manganese sulfate monohydrate, 6×10⁻⁷ M of sodium selenite, 0.5×10⁻³ M of sodium metasilicate, 5×10⁻⁶ M of ammonium metavanadate, 5×10⁻¹⁰ M of nickel chloride hexahydrate, 5×10⁻¹⁰ M of stannous chloride dihydrate, 5×10⁻⁷ M of ethanolamine, 5×10⁻⁶ M of O-phosphorylethanolamine, 3×10⁻⁹ M of ammonium molybdate tetrahydrate, 5.4 g/L of 4-(2-hydroxyethyl) piperazine-1-ethanesulfonic acid4-(2-hydroxyethyl) piperazine-1-ethanesulfonic acid, 30 μg/mL of vitamin C, 2% of the bovine serum albumin, 1% of the lipid concentrate, and 10% of the serum substitute.

Preferably, the serum-free culture medium further includes an antibiotic.

More preferably, the antibiotic is a penicillin-streptomycin solution.

More further preferably, an addition amount of penicillin is 100 IU/mL, and an addition amount of streptomycin is 100 IU/mL.

More further preferably, 5 mL of the 100×penicillin-streptomycin solution is added into every 500 mL of the serum-free culture medium.

Preferably, the basic medium is a DMEM/Ham's F12 medium.

More preferably, a volume ratio of the DMEM to Ham's F12 is 1:1 in the basic medium.

In the present invention, the serum substitute refers to a fully artificially synthesized stem cell medium ingredient without animal-derived protein pollution and suitable for clinical researches. The serum substitute according to the present invention is purchased from Invitrogen under No. 10828-028.

In the present invention, the lipid concentrate refers to a concentrated lipid mixture with specific chemical ingredients, is a greasy supplement for serum-free culture of vertebrate cells and non-vertebrate cells, and plays an important role in cell structure and energy supply. The lipid concentrate, i.e., lipid concentrate with specific chemical ingredients, is purchased from Gibco under No. 11905031.

Accordingly, use of the serum-free culture medium in vitro culture of limbal stem cells also falls within the scope of protection of the present invention.

The present invention further provides an isolation and culture method of limbal stem cells. The isolation and culture method includes the steps of: washing a corneal limbus tissue, enzymolyzing the corneal limbus tissue, and culturing a product after the enzymolyzing in the serum-free culture medium for limbal stem cells.

Specifically, the isolation and culture method according to the present invention includes: taking a corneal limbus tissue of a human fetus dying within 48 hours, washing the corneal limbus tissue with a PBS containing a penicillin-streptomycin solution, enzymolyzing the corneal limbus tissue, culturing a product after the enzymolyzing in the serum-free culture medium for limbal stem cells, and subculturing.

Preferably, the enzymolyzing is one-time enzymolyzing. And the enzymolyzing is carried out directly using a type IV collagenase. According to the present invention, the type IV collagenase can be used for enzymolysis and isolation, so that the corneal limbus tissue is fully enzymolyzed in the presence of collagenase, thereby ensuring that individual cells are obtained, damage to cells is reduced, and the limbal stem cells yield is improved.

Preferably, a concentration of the type IV collagenase is 0.1%-0.5%, this means that the enzymolyzing is carried out using 0.1%-0.5% of the type IV collagenase; and the enzymolyzing lasts for 30-60 minutes.

More preferably, the enzymolyzing is carried out using 0.2% of the type IV collagenase; the type IV collagenase is dissolved in the DMEM/Ham's F12 medium; and the enzymolyzing lasts for 45 minutes.

Preferably, according to the isolation and culture method of limbal stem cells, before culturing the product after the enzymolyzing in the serum-free culture medium, a collagen I is first added, and then isolated limbal stem cells are added.

According to the present invention, the collagen I is used for coating culture plates or other materials as a substrate for limbal stem cell growth, so as to improve the purity and stability of limbal stem cells, and contribute to cell adherence.

Specifically, preferably, the isolation and culture method of limbal stem cells includes the following steps:

S1. taking a corneal limbus tissue of a human fetus dying within 48 hours, washing the corneal limbus tissue with a PBS containing a penicillin-streptomycin solution, enzymolyzing the corneal limbus tissue with 0.2% of a type IV collagenase for 45 minutes, terminating the enzymolysis by adding the serum-free culture medium according to the present invention, centrifuging, and discarding the supernatant; and

S2. cooling 10% collagen I in an ice bath, coating a culture dish with the 10% collagen I after cooling, adding the serum-free culture medium containing isolated limbal stem cells into the culture dish, resuspending cells, and then culturing the cells in the serum-free culture medium. When limbal stem cells are isolated using the culture medium provided by the present invention, a positive rate of antibodies, such as p63 and PAX6, in the cells is 96%-100%.

Compared with the prior art, the present invention has the following beneficial effects:

(1) The medium ingredients provided by the present invention are free of serum, and can provide necessary adequate nutrition and good environment for limbal stem cells growth and proliferation; the supplements can effectively replace serum ingredients through various mechanisms in the process of culturing limbal stem cells, and realize favorable cell growth; and the limbal stem cells have fast proliferation, high purity, high activity and good stability in the culture process.

(2) The present invention has established a novel limbal stem cell culture method free of trophoblast, carrier and serum, so as to obtain homogeneous limbal stem cells with an efficient in vitro amplification function, improve the cell purity, improve the cell quality, and provide quick and stable cell sources for researches on the mechanism of limbal stem cell specificity and transplantation therapy.

### BRIEF DESCRIPTION OF THE DRAWINGS

FIG. 1 shows a serum-free culture of human limbal stem cells (morphological features under a microscope).

FIG. 2A to FIG. 2C show an immunochemical staining of limbal stem cells specifically labeled with PAX6 antibody; wherein FIG. 2A is DAPI; FIG. 2B is PAX6; and FIG. 2C is after fusion.

FIG. 3A to FIG. 3C show an immunochemical staining of limbal stem cells specifically labeled with p63 antibody; wherein FIG. 3A is DAPI; FIG. 3B is p63; and FIG. 3C is after fusion.

FIG. 4A to FIG. 4C show immunochemical staining of limbal stem cells specifically labeled with Ki67 antibody; wherein FIG. 4A is DAPI; FIG. 4B is Ki67; and FIG. 4C is after fusion.

FIG. 5A to FIG. 5C show immunochemical staining of limbal stem cells specifically labeled with K12 antibody; wherein FIG. 5A is DAPI; FIG. 5B is K12; and FIG. 5C is after fusion.

FIG. 6 shows morphological features of cells cultured in the serum-free culture medium according to the present invention for 4 days.

FIG. 7 shows morphological features of cells cultured in the serum-free culture medium according to the present invention for 8 days.

FIG. 8 shows morphological features of cells cultured in the serum-free culture medium according to the present invention for 20 days.

FIG. 9 shows morphological features of P1 generation LSCs subcultured in the serum-free culture medium according to the present invention.

FIG. 10 shows an FBS-containing culture medium for human limbal stem cells (morphological features under a microscope).

### DESCRIPTION OF THE EMBODIMENTS

The present invention is further described below in combination with specific examples, but the examples do not limit the present invention in any way. Without departing from the spirit and essence of the present invention, all simple modifications or replacements of the methods, steps or conditions of the present invention fall within the scope of the present invention. Unless otherwise specified, the technical means used in the examples are the conventional means known to those skilled in the art.

Unless otherwise stated, all reagents and materials used in the following examples are available on the market.

Collagen I: dissolving 1 mL of a collagen I in 9 mL of DMEM/Ham's F12 medium.

Type IV collagenase: 5 mg/mL of a type IV collagenase prepared from DMEM/Ham's F12 medium.

Stock solution of human recombinant EGF: 10 μg/mL EGF stock solution prepared from PBS.

Stock solution of insulin: prepared from 0.005 mol/L HCl, with a concentration of 5 mg/mL.

Stock solution of 3,3′,5-triiodo-L-thyronine: dissolving 13.6 mg of 3,3′,5-iodothyronine in 15 mL of 0.02 mol/L NaOH solution, adding 85 mL of PBS; and diluting 0.1 mL of the prepared solution with PBS to 20 mL as the stock solution with a concentration of 1×10⁻⁶ M.

Stock solution of hydrocortisone: dissolving 5 mg of hydrocortisone in 1 mL of anhydrous ethanol, and adding 400 μL of the resulting solution to 10 mL of PBS to obtain the stock solution.

Stock solution of manganese sulfate monohydrate: dissolving 1.69 mg of manganese sulfate monohydrate in 10 mL of PBS to obtain a 10⁶-fold stock solution at a concentration of 1×10⁻³ M.

Stock solution of sodium metasilicate: dissolving 0.61 g of sodium metasilicate in 10 mL of PBS to obtain a 1000-fold stock solution at a concentration of 0.5 M.

Stock solution of sodium selenite: dissolving 1.038 g of sodium selenite in 10 mL of PBS to obtain a 10⁶-fold stock solution at a concentration of 0.6 M.

Stock solution of ammonium metavanadate: dissolving 5.85 g of ammonium metavanadate in 10 mL of PBS to obtain a 10⁶-fold stock solution at a concentration of 5 M.

Stock solution of nickel chloride hexahydrate: dissolving 1.19 mg of nickel chloride hexahydrate in 10 mL of PBS to obtain a 10⁶-fold stock solution at a concentration of 5×10⁻⁴ M.

Stock solution of stannous chloride dihydrate: dissolving 1.13 mg of stannous chloride dihydrate in 10 mL of PBS to obtain a 10⁶-fold stock solution at a concentration of 5×10⁻⁴ M.

Stock solution of ethanolamine: dissolving 305 mg of ethanolamine in 10 mL of PBS to obtain a 10⁶-fold stock solution at a concentration of 0.5 M.

Stock solution of O-phosphoiylethanolamine: dissolving 7 mg of O-phosphorylethanolamine in 10 mL of PBS to obtain a 1000-fold stock solution at a concentration of 5×10⁻³ M.

Stock solution of ammonium molybdate tetrahydrate: dissolving 37.08 mg of ammonium molybdate tetrahydrate in 10 mL of PBS to obtain a 10⁶-fold stock solution at a concentration of 3×10⁻³ M.

Stock solution of vitamin C: dissolving 300 mg of vitamin C in 10 mL of PBS to obtain a 1000-fold stock solution at a concentration of 30 mg/mL.

### EXAMPLE 1

1. A Serum-Free Culture Medium for Limbal Stem Cells

A serum-free culture medium for limbal stem cells consists of following ingredients:

216 mL of a Ham's F12, 216 mL of a DMEM, 5 mL of 100×penicillin-streptomycin solution, 1 mL of a stock solution of a human recombinant EGF, 500 μL of a stock solution of insulin, 500 μL of a stock solution of vitamin C, 500 μL of a stock solution of 3,3′,5-triiodo-L-thyronine, 500 μL of a stock solution of hydrocortisone, 1 μL of a stock solution of a forskolin, 0.5 μL of manganese sulfate monohydrate, 0.5 μL of sodium selenite, 500 μL of sodium metasilicate, 0.5 μL of ammonium metavanadate, 0.5 μL of nickel chloride hexahydrate, 0.5 μL of stannous chloride dihydrate, 0.5 μL of ethanolamine, 500 μL of O-phosphorylethanolamine, 0.5 μL of ammonium molybdate tetrahydrate, 2.7 g of 4-(2-hydroxyethyl) piperazine-1-ethanesulfonic acid, 10 g of a bovine serum albumin, 5 mL of a lipid concentrate, and 50 mL of a serum substitute.

Concentrations of supplements are respectively: 10 ng/mL of the human recombinant EGF, 5 μg/mL of insulin, 2.5×10⁻⁹ M of 3,3′,5-triiodo-L-thyronine, 0.4 μg/mL of hydrocortisone, 1×10⁻⁵ M of the forskolin, 1×10⁻⁹ M of manganese sulfate monohydrate, 6×10⁻⁷ M of sodium selenite, 0.5×10⁻³ M of sodium metasilicate, 5×10⁻⁶ M of ammonium metavanadate, 5×10⁻¹⁰ M of nickel chloride hexahydrate, 5×10⁻¹⁰ M of stannous chloride dihydrate, 5×10⁻⁷ M of ethanolamine, 5×10⁻⁶ M of O-phosphorylethanolamine, 3×10⁻⁹ M of ammonium molybdate tetrahydrate, 5.4 g/L of 4-(2-hydroxyethyl) piperazine-1-ethanesulfonic acid, 30 μg/mL of vitamin C, 2% of the bovine serum albumin, 1% of the lipid concentrate, and 10% of the serum substitute.

2. An Isolation and Culture Method of Limbal Stem Cells

(1) Under an operating microscope, a human corneal limbus tissue was cut with a tissue forcep and corneal scissors, and washed with a PBS containing a penicillin-streptomycin solution (1×) in a sterile environment twice (5 minutes each time); and then cut into pieces with the scissors.

(2) According to a volume of a tissue block, 5 mL of 0.5% type IV collagenase solution was added to every 1 cm³ of the tissue block, digested while gently oscillating at 37° C. for 45 minutes, neutralized with 10-fold volume of the serum-free culture medium, and centrifuged at 1000 rpm for 5 minutes. Then the supernatant was discarded.

(3) After a 12-well plate was coated with 10% matrigel, centrifuged cells were resuspended in the serum-free culture medium, respectively cultivated in the wells of the 12-well plate, and cultured in an incubator at 37° C. under an atmosphere containing 5% CO₂. The solution was replaced every other day, and the growth status of the cells was observed under a microscope.

3. Analysis on the Experimental Results:

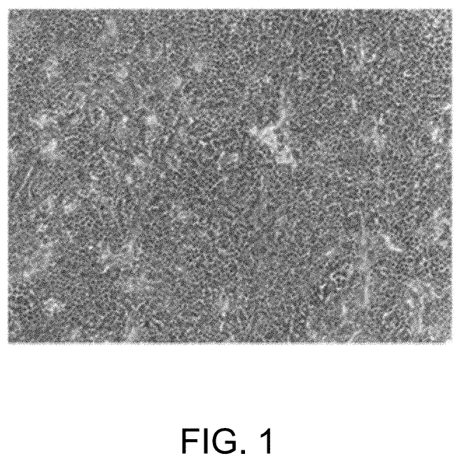

(1) FIG. 1 shows serum-free culture of human limbal stem cells. The results showed that the cultivated cells have the morphological structure of limbal stem cells, proving that the limbal stern cells can really be cultured in the serum-free culture medium.

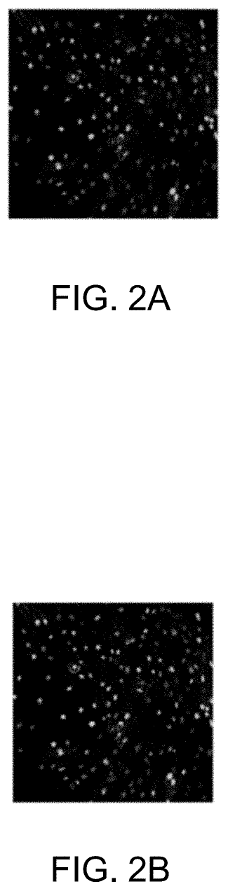

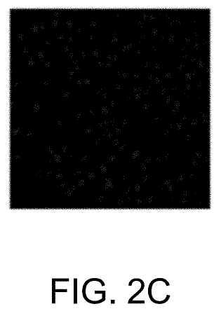

(2) FIGS. 2A, 2B and 2C show immunochemical staining of limbal stem cells specifically labeled with PAX6 antibody; wherein blue is DAPI, and green is PAX6. The results show that the cultured cells are PAX6 positive, have the characteristics of limbal stem cells, and have a PAX6 antibody positive rate of 96%-100%.

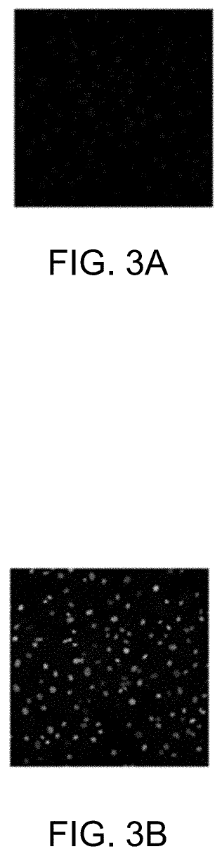

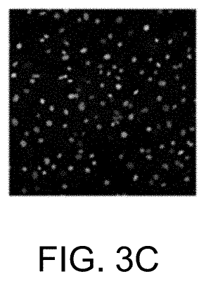

(3) FIGS. 3A, 3B and 3C show immunochemical staining of stem cells specifically labeled with p63 antibody; wherein blue is DAPI, and green is p63. The results show that the cultured cells are p63 positive, have the characteristics of stem cells, and have a p63 antibody positive rate of 96%-100%.

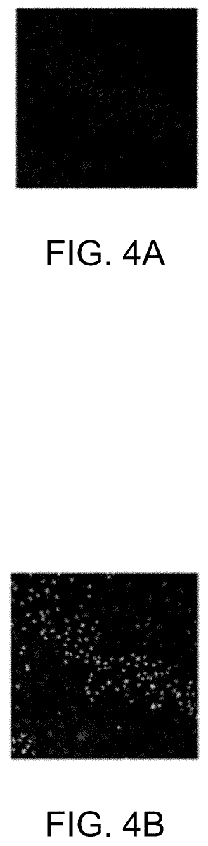

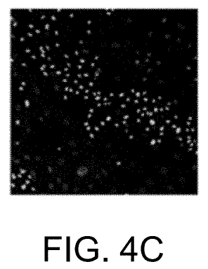

(4) FIGS. 4A, 4B and 4C show immunochemical staining of limbal stem cells specifically labeled with Ki67 antibody; wherein blue is DAPI, and green is Ki67. The results show that the cultured cells are Ki67 positive, have the characteristics of limbal stem cells, and have a Ki67 antibody positive rate of 96%-100%.

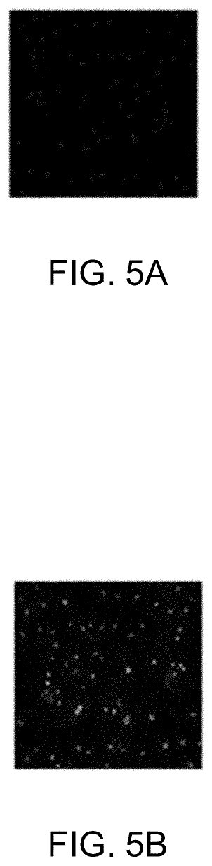

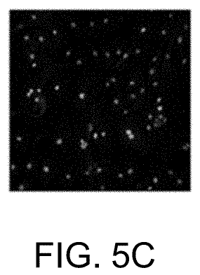

(5) FIGS. 5A, 5B and 5C show immunochemical staining of limbal stem cells specifically labeled with K12 antibody; wherein blue is DAPI, and green is K12. The results show that the cultured cells are K12 positive, have the characteristics of limbal stem cells, and have a K12 antibody positive rate of 96%-100%.

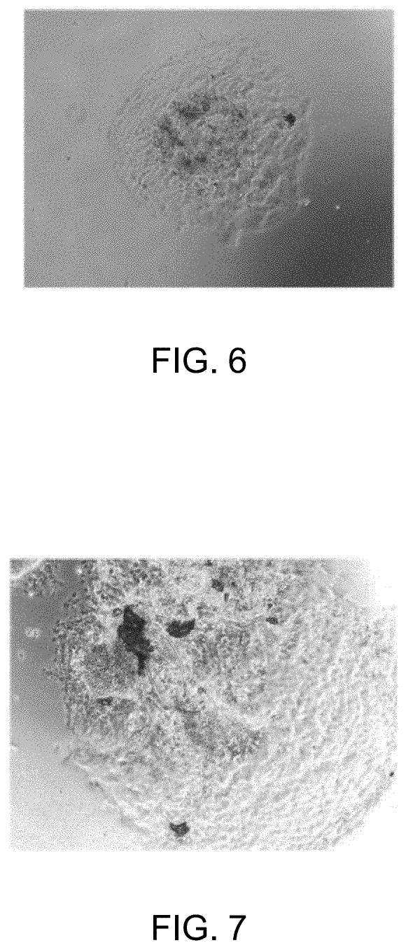

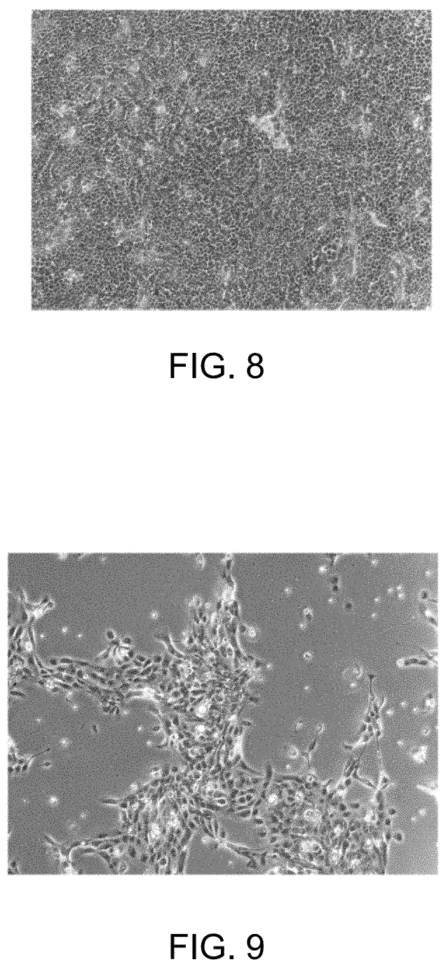

(6) FIGS. 6-9 show the morphological features of cells cultured in the serum-free culture medium according to the present invention for 4-20 days and the morphological features of subcultured cells. The results show that the morphological features of cells comply with the characteristics of limbal stem cells.

As can be concluded from the above results, the serum-free culture medium according to the present invention can replace a serum-containing medium very well, and realize limbal stem cells culture and favorable cells growth. In the culture process, the limbal stem cells have fast proliferation, high purity, high activity and good stability; homogeneous limbal stem cells with an efficient in vitro amplification function have been obtained; and the cells quality has been improved, thereby providing a more quick and stable cell source for researches on the mechanism of limbal stem cell specificity and transplantation therapy.

### EXAMPLE 2

### Culture Medium Formula

The experimental method is identical to that in Example 1 only except that the concentrations of the supplements in the serum-free culture medium for limbal stem cells in the example are as follows:

10 ng/mL of the human recombinant EGF, 10 μg/mL of insulin, 5×10⁻⁹ M of 3-iodothyronine, 0.2 μg/mL of hydrocortisone, 2×10⁻⁵ M of the forskolin, 0.5×10⁻⁹ M of manganese sulfate monohydrate, 5×10⁻⁷ M of sodium selenite, 0.1×10⁻³ M of sodium metasilicate, 8×10⁻⁶ M of ammonium metavanadate, 3×10⁻¹⁰ M of nickel chloride hexahydrate, 8×10⁻¹⁰ M of stannous chloride dihydrate, 3×10⁻⁷ M of ethanolamine, 8×10⁻⁶ M of O-phosphorylethanolamine, 1×10⁻⁹ M of ammonium molybdate tetrahydrate, 8 g/L of 4-(2-hydroxyethyl) piperazine-1-ethanesulfonic acid, 20 μg/mL of vitamin C, 3% of the bovine serum albumin, 0.5% of lipid concentrate, and 15% of the serum substitute.

### EXAMPLE 3

### Culture Medium Formula

The experimental method is identical to that in Example 1 only except that the concentrations of the supplements in the serum-free culture medium for limbal stem cells in the example are as follows:

20 ng/mL of the human recombinant EGF, 5 μg/mL of insulin, 1×10⁻⁹ M of 3-iodothyronine, 1 μg/mL of hydrocortisone, 0.5×10⁻⁵ M of the forskolin, 2×10⁻⁹ M of manganese sulfate monohydrate, 10×10⁻⁷ M of sodium selenite, 1×10⁻³ M of sodium metasilicate, 3×10⁻⁶ M of ammonium metavanadate, 8×10⁻¹⁰ M of nickel chloride hexahydrate, 3×10⁻¹⁰ M of stannous chloride dihydrate, 8×10⁻⁷ M of ethanolamine, 3×10⁻⁶ M of O-phosphorylethanolamine, 6×10⁻⁹ M of ammonium molybdate tetrahydrate, 2 g/L of 4-(2-hydroxyethyl) piperazine-1-ethanesulfonic acid, 50 μg/mL of vitamin C, 1% of the bovine serum albumin, 2% of the lipid concentrate, and 5% of the serum substitute.

### COMPARISON EXAMPLE 1

### Positive Control (Culture Medium Containing Fetal Bovine Serum)

The experimental method is identical to that in Example 1 only except that the medium for limbal stem cells in the example contains serum, and specifically includes the following ingredients: 216 mL of the Ham's F12, 216 mL of the DMEM, 5 mL of 100×penicillin-streptomycin solution and 63 mL of the fetal bovine serum.

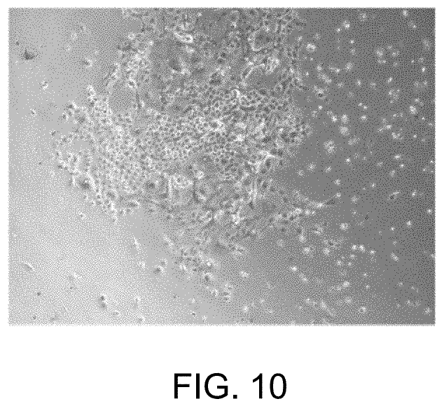

FIG. 10 shows a serum-containing culture of human limbal stem cells. It is found through researches that the limbal stem cells obtained from the serum-free culture medium in Example 1 are not significantly different from those obtained from the serum-containing medium; and the limbal stem cells in the serum-free culture medium have almost equivalent growth rate to those in the serum-containing medium, and have regular and uniform morphological features, suggesting that the limbal stem cells cultured in the serum-free culture medium according to the present invention also have very high purity. The results show that the serum-free culture medium can completely replace the serum-containing medium in culturing limal stem cells.

### COMPARISON EXAMPLE 2

### Negative Control (Only Containing a Basic Medium and Antibiotics)

The experimental method is identical to that in Example 1 only except that the medium for limbal stem cells in the example contains 216 mL of the Ham's F12, 216 mL of the DMEM, and 5 mL of 100×penicillin-streptomycin solution.

It is found through researches that compared with the serum-free culture medium in Example 1, the limbal stem cells completely fail to grow adherently. The results show that this medium is not suitable for culturing limbal stem cells.

### COMPARISON EXAMPLE 3

### Medium Containing a Basic Medium, Antibiotics and a Serum Substitute

The experimental method is identical to that in Example 1 only except that the medium for limbal stem cells in the example contains 216 mL of the Ham's F12, 216 mL of the DMEM, 5 mL of 100×penicillin-streptomycin solution and 10% of the serum substitute.

The results show that compared with the serum-free culture medium in Example 1, there is poor limbal stem cells growth status. Therefore, the medium is not suitable for culturing limbal stem cells.

### COMPARISON EXAMPLE 4

The conditions are identical to those in Example 1, except that a concentration of the serum substitute is 1%.

The results show that when the concentration of the serum substitute is 1%, and the concentration of the serum substitute is very low, which can lead morphological features of the limbal stem cells to changing.

### COMPARISON EXAMPLE 5

The conditions are identical to those in Example 1, except that a concentration of forskolin is 0.1×10⁻⁵ M.

The results show that when the serum concentration is reduced from 1×10⁻⁵ M to 0.1×10⁻⁵ M, the limbal stem cells become larger, their cell nuclei become smaller, and their cell adherence capacity becomes worse, etc.

### COMPARISON EXAMPLE 6

The conditions are identical to those in Example 1, except that a concentration of the lipid concentrate is 0.1%.

The results show that the concentration of the lipid concentrate is 0.1%, which can lead the limbal stem cells to growing slowly, the epidermal cells doing not have obvious morphological features, and there are loose intercellular connections of limbal stem cells and many dead cells in the serum-free culture medium.

Furthermore, it is found that the human recombinant EGF, insulin, 3-iodo-L-thyronine, hydrocortisone, the forskolin, manganese sulfate monohydrate, sodium selenite, sodium metasilicate, ammonium metavanadate, nickel chloride hexahydrate, stannous chloride dihydrate, ethanolamine, O-phosphorylethanolamine, ammonium molybdate tetrahydrate, 4-(2-hydroxyethyl) piperazine-1-ethanesulfonic acid, vitamin C, the bovine serum albumin, the lipid concentrate and the serum substitute all play an important role in culturing limbal stem cells, and none thereof is dispensable.

What is claimed is:

1. A serum-free culture medium for limbal stem cells, comprising a basic medium and supplements; wherein the supplements comprise following ingredients by concentration: 10-20 ng/mL of an human recombinant EGF, 5-10 μg/mL of insulin, 1×10⁻⁹-5×10⁻⁹ M of 3,3′,5-triiodo-L-thyronine, 0.2-1 μg/mL of hydrocortisone, 0.5×10⁻⁵-2×10⁻⁵ M of a forskolin, 0.5×10⁻⁹-2×10⁻⁹ M of manganese sulfate monohydrate, 5×10⁻⁷-10×10⁻⁷ M of sodium selenite, 0.1×10⁻³-1×10⁻³ M of sodium metasilicate, 3×10⁻⁶-8×10⁻⁶ M of ammonium metavanadate, 3×10⁻¹⁰-8×10⁻¹⁰ M of nickel chloride hexahydrate, 3×10⁻¹⁰-8×10⁻¹⁰ M of stannous chloride dihydrate, 3×10⁻⁷-8×10⁻⁷ M of ethanolamine, 3×10⁻⁶-8×10⁻⁶ M of O-phosphorylethanolamine, 1×10⁻⁹-6×10⁻⁹ M of ammonium molybdate tetrahydrate, 2-8 g/L of 4-(2-hydroxyethyl) piperazine-1-ethanesulfonic acid, 20-50 μg/mL of vitamin C, 1%-3% of a bovine serum albumin, 0.5%-2% of a lipid concentrate, and 10%-15% of a serum substitute.

2. The serum-free culture medium for limbal stem cells according to claim 1, wherein concentrations of the ingredients in the supplements of the serum-free culture medium are as follows: 10 ng/mL of the human recombinant EGF, 5 μg/mL of insulin, 2.5×10⁻⁹ M of 3,3′,5-triiodo-L-thyronine, 0.4 μg/mL of hydrocortisone, 1×10⁻⁵ M of the forskolin, 1×10⁻⁹ M of manganese sulfate monohydrate, 6×10⁻⁷ M of sodium selenite, 0.5×10⁻³ M of sodium metasilicate, 5×10⁻⁶ M of ammonium metavanadate, 5×10⁻¹⁰ M of nickel chloride hexahydrate, 5×10⁻¹⁰ M of stannous chloride dihydrate, 5×10⁻⁷ M of ethanolamine, 5×10⁻⁶ M of O-phosphorylethanolamine, 3×10⁻⁹ M of ammonium molybdate tetrahydrate, 5.4 g/L of 4-(2-hydroxyethyl) piperazine-1-ethanesulfonic acid, 30 μg/mL of vitamin C, 2% of the bovine serum albumin, 1% of the lipid concentrate, and 10% of the serum substitute.

3. The serum-free culture medium for limbal stem cells according to claim 1, wherein the serum-free culture medium further comprises an antibiotic.

4. The serum-free culture medium for limbal stem cells according to claim 3, wherein the antibiotic is a penicillin-streptomycin solution.

5. The serum-free culture medium for limbal stem cells according to claim 1, wherein the basic medium is a DMEM/Ham's F12 medium.

6. The serum-free culture medium for limbal stem cells according to claim 5, wherein a volume ratio of the DMEM to Ham's F12 is 1:1.

7. An isolation and culture method of limbal stem cells, comprising the steps of: washing a corneal limbus tissue, enzymolyzing the corneal limbus tissue, and culturing a product after the enzymolyzing in the serum-free culture medium, wherein the serum-free culture medium comprising a basic medium and supplements, the supplements comprise following ingredients by concentration: 10-20 ng/mL of an human recombinant EGF, 5-10 μg/mL of insulin, 1×10⁻⁹-5×10⁻⁹ M of 3,3′,5-triiodo-L-thyronine, 0.2-1 μg/mL of hydrocortisone, 0.5×10⁻⁵-2×10⁻⁵ M of a forskolin, 0.5×10⁻⁹-2×10⁻⁹ M of manganese sulfate monohydrate, 5×10⁻⁷-10×10⁻⁷ M of sodium selenite, 0.1×10⁻³-1×10⁻³ M of sodium metasilicate, 3×10⁻⁶-8×10⁻⁶ M of ammonium metavanadate, 3×10⁻¹⁰-8×10⁻¹⁰ M of nickel chloride hexahydrate, 3×10⁻¹⁰-8×10⁻¹⁰ M of stannous chloride dihydrate, 3×10⁻⁷-8×10⁻⁷ M of ethanolamine, 3×10⁻⁶-8×10⁻⁶ M of O-phosphorylethanolamine, 1×10⁻⁹-6×10⁻⁹ M of ammonium molybdate tetrahydrate, 2-8 g/L of 4-(2-hydroxyethyl) piperazine-1-ethanesulfonic acid, 20-50 μg/mL of vitamin C, 1%-3% of a bovine serum albumin, 0.5%-2% of a lipid concentrate, and 10%-15% of a serum substitute.

8. The isolation and culture method according to claim 7, wherein the enzymolyzing is one-time enzymolyzing, and the enzymolyzing is carried out directly using a type IV collagenase.

9. The isolation and culture method according to claim 8, wherein a concentration of the type IV collagenase is 0.1%-0.5%; and the enzymolyzing lasts for 30-60 minutes.
